(19) United States
(12) Patent Application Publication
(10) Pub. No.: US 2014/0288189 A1
(43) Pub. Date: Sep. 25, 2014

(54) Water-Stable Hydrogel and Method Using Same

(71) Applicants: Arizona Board of Regents on behalf of Arizona State University, Scottsdale, AZ (US); Banner Health, Phoenix, AZ (US)

(72) Inventors: Derek Overstreet, Chandler, AZ (US); Brent Vernon, Queen Creek, AZ (US); Ryan McLemore, Phoenix, AZ (US)

(21) Appl. No.: 14/351,551

(22) PCT Filed: Oct. 12, 2012
(86) PCT No.: PCT/US12/60121

§ 371 Date: Apr. 11, 2014

Related U.S. Application Data
(60) Provisional application No. 61/546,397, filed on Oct. 12, 2011.

(51) Int. Cl. A61K 47/32; A61K 9/00

(52) U.S. Cl. CPC A61K 47/32; A61K 9/0019
USPC 514/772.1; 525/329.4

(57) ABSTRACT

A hydrogel (I) comprising a polymer backbone comprising a plurality of repeat units, wherein one or more of the repeat units comprises a pendent water soluble polymer attached thereto by a linkage selected from the group consisting of amide, thioamide, urea, and thiourea.

### CROSS-REFERENCE TO RELATED APPLICATIONS

This application is based on, claims a priority benefit from, and incorporates herein by reference U.S. Patent Application No. 61/546,397, filed Oct. 12, 2011, and entitled, “Water-Stable Non-Ionic Hydrogel and Method Using Same.”

### BACKGROUND OF THE INVENTION

In situ forming hydrogels are useful for a variety of biological and biomedical applications including drug delivery, embolization, cell encapsulation and culture, and tissue regeneration.

For most applications where in situ forming hydrogels are used, resistance to shrinking is an important consideration. Usually, the ideal case is that the material transitions quickly from liquid to solid with almost no change in volume. Shrinking or swelling inherently causes changes in the hydrogel's mechanical properties, porosity, and size. Wound healing and embolization applications require retention of the hydrogel's original size at the injection site and good contact with the surrounding tissue. For controlled drug delivery, a fast sol-to-gel transition without syneresis could reduce the high initial burst release of hydrophilic drugs typical of many in situ forming materials. For successful use as synthetic extracellular matrices in vitro or in vivo, gels must retain a high volume fraction of water in order to support cell growth.

### SUMMARY OF THE INVENTION

Applicants' hydrogel composition comprises a polymeric backbone comprising a plurality of first repeat units in combination with one or more second repeat units each comprising a water soluble polymer attached thereto by a linkage selected from the group consisting of amide, thioamide, urea, and thiourea In certain embodiments, the first repeat units comprise a substituted acrylamide. In certain embodiments, the water soluble polymer comprises a polyether. The water soluble polymer increases gel swelling and significantly slows the release of entrapped drugs with a very minor effect on the graft copolymer's lower critical solution temperature (“LCST”) in physiological buffers. Compared to other polymeric hydrogels comprising hydrophilic repeat units such as PEG-acrylates and acrylic acid, Applicants' substituted polyacrylamide backbone includes water-stable linkages comprising one or more polyethers. Applicants' hydrogel comprises water-stable pendent linkages rather than pendent ester moieties that degrade within a time frame of hours to days. Applicants' graft copolymer is useful as an injectable drug delivery vehicle, and also comprises a platform from which a variety of derivative materials can be prepared where the swelling and/or drug release can be tuned almost independently of the LCST properties, which usually are greatly affected by comonomers which maintain or increase gel swelling.

Applicants have found that these design principles can be utilized to prepare multiple embodiments of their graft copolymer wherein the polymer backbone controls sensitivity to the environment (a so-called “smart” material) and the pendent side chains independently control other properties such as drug delivery, swelling, and chemical reactivity.

### BRIEF DESCRIPTION OF THE DRAWINGS

FIG. 1. 1H NMR spectra of high molecular weight poly(NIPAAm) and poly(NIPAAm-co-JAAm) in D2O, wherein JAAm is JEFFAMINE M-1000 acrylamide and NIPAAm is N-isopropylacrylamide.

FIG. 2. Differential scanning calorimetry thermograms for 5 wt % solutions of (A) high molecular weight (“HMW”) and (B) low molecular weight (“LMW”) copolymers of poly(NIPAAm-co-JAAm) in 150 mM PBS, pH 7.4;

FIG. 3. Relative absorbance as a fraction of the maximum absorbance of each sample (λ=450 nm) of 0.1 wt % solutions of synthesized copolymers in 150 mM PBS, pH 7.4. The dotted vertical line denotes body temperature;

FIG. 4. Percentage of initial gel volume at various times after gelation for (A) HMW and (B) LMW copolymers of poly(NIPAAm-co [symbol] -JAAm) at various polymer concentrations. Error bars represent one standard deviation (n=3);

FIG. 5. Gel swelling of 20 wt % H 0 (top row) and H 30 (bottom row) at 30 min (a, d), 1 day (b, e), and 42 days (c, f) after gelation at 37° C., wherein the number after the letter denotes the JAAm percentage in the feed relative to NIPAAm;

FIG. 6. Solution viscosity at 20° C. of 20 H 0 (squares) and 20 H 30 (circles) as a function of shear rate;

FIG. 7. Storage (G′) and loss (G″) moduli of 20 H 0 and 20 H 30 subjected to oscillatory frequency sweeps with active normal force control at 37° C. Error bars represent one standard deviation (n=4);

FIG. 8. Cumulative fraction of ovalbumin released from 20 H 0 (squares) and 20 H 30 (triangles) hydrogels at 37° C. in 150 mM PBS, pH 7.4. Error bars represent one standard deviation (n=3). Some error bars are smaller than the data points;

FIG. 9 is a table describing Applicants' terpolymer hydrogel compositions; and

FIG. 10 is a flow chart summarizing Applicants' method using Applicants' copolymer hydrogel.

### DETAILED DESCRIPTION OF PREFERRED EMBODIMENTS

Referring to the foregoing paragraphs, this invention is described in preferred embodiments in the following description with reference to the Figures, in which like numerals represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of the invention may be combined in any suitable manner in one or more embodiments. In the above description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

Biological and medical applications of in situ forming hydrogels often require control over swelling without affecting other functionalities such as affecting the solution-to-gel transition conditions of the material. Toward this end, Applicants have prepared temperature-responsive graft copolymer I, wherein R1 and R2 are independently selected from the group consisting of H, alkyl, phenyl, benzyl, 2-cyanoprop-2-yl, 4-cyanopentanoic acid-4-ylethyl-2-propionate, sulfate, 2-[2-methoxypropan-2-yl)oxy]propan-2-yl, and a dithioester derived from a RAFT chain transfer agent such as 4-cyano-4-(ethylsulfanylthiocarbonyl) sulfanylpentanoic acid. R3 and R4 are each independently selected from the group consisting of H, methyl, ethyl, and phenyl.

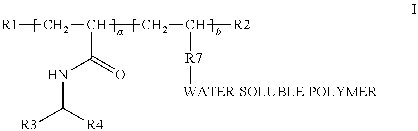

In certain embodiments, R7 comprises an amide linkage. In certain embodiments, R7 comprises a thioamide linkage. In certain embodiments, R7 comprises a urea linkage. In certain embodiments, R7 comprises a thiourea linkage.

In certain embodiments, the water soluble polymer comprises a polyether. In certain embodiments, the water soluble polymer comprises polyether VI formed by ring opening polymerization of ethylene oxide, wherein R6 is selected from the group consisting of H, methyl, methoxy, and hydroxyl. In certain embodiments, n is between about 5 and about 2500.

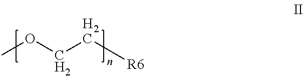

In certain embodiments, the water soluble polymer comprises polyether VII formed by ring opening polymerization of propylene oxide. In certain embodiments, n is between about 15 and about 250.

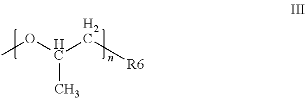

In certain embodiments, the water soluble polymer comprises polyether VIII formed by co-polymerization of ethylene oxide and propylene oxide. In certain embodiments, r is between about 5 and about 2500, and p is between about 1 and about 1000.

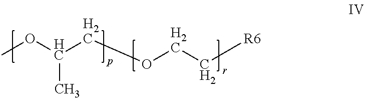

In certain embodiments, the water soluble polymer comprises polyether IX formed by ring opening polymerization of tetrahydrofuran. In certain embodiments, n is between about 10 and about 50.

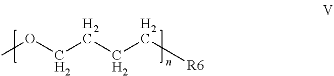

In certain embodiments, the water soluble polymer comprises a water-soluble polymer of one or more of the following: vinyl alcohol, acrylic acid, methacrylic acid, 2-hydroxyethyl methacrylate, N-2 hydroxypropylmethacrylamide, vinylpyrrolidone, or a monosaccharide.

In certain embodiments, graft copolymer I comprises a copolymer comprising a plurality of repeat units VI and a plurality of repeat units VIII formed by copolymerizing monomers VII and IX, wherein R7 comprises an amide linkage.

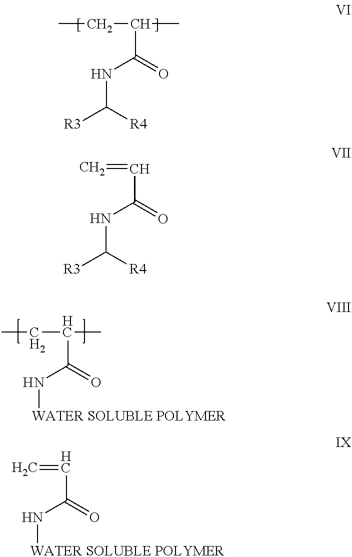

In certain embodiments, graft copolymer I comprises a copolymer comprising a plurality of repeat units VI and a plurality of repeat units X formed by copolymerizing monomer VII and monomer XI, wherein R7 comprises a thioamide linkage.

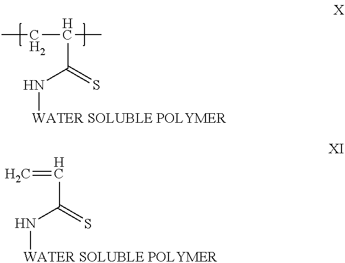

In certain embodiments, graft copolymer I comprises a copolymer comprising a plurality of repeat units VI and a plurality of repeat units XII formed by copolymerizing monomer VII and monomer XIII, wherein R7 comprises a urea linkage.

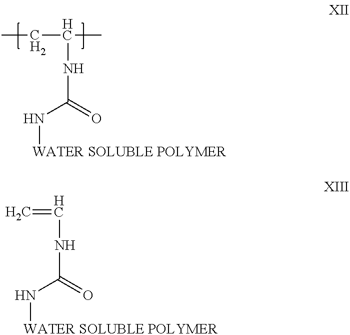

In certain embodiments, graft copolymer I comprises a copolymer comprising a plurality of repeat units VI and a plurality of repeat units XIV formed by copolymerizing monomer VII and monomer XV, wherein R7 comprises a thiourea linkage.

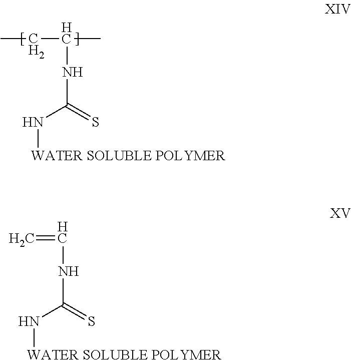

In certain embodiments, a is between about 10 and about 10000, b is between about 1 and about 1000. Graft copolymer I can be synthesized via a number of different procedures. For example, graft copolymer I can be prepared by free radical polymerization.

Graft copolymer I can also be prepared by reversible addition-fragmentation chain transfer (“RAFT”) polymerization. Those skilled in the art will appreciate that a RAFT polymerization can be performed by adding a quantity of a RAFT agent (thiocarbonylthio compounds) to a conventional free radical polymerization. Usually the same monomers, initiators, solvents and temperatures can be used. Because of the low concentration of the RAFT agent in the system, the concentration of the initiator is usually lower than in conventional radical polymerization. Radical initiators such as azobisisobutyronitrile (AIBN) and 4,4′-Azobis(4-cyanovaleric acid) (ACVA) which is also called 4,4′-Azobis(4-cyanopentanoic acid) are widely used as the initiator in RAFT. RAFT polymerization is known for its compatibility with a wide range of monomers, including for example acrylates and acrylamides.

Graft copolymer I, as either a random copolymer or a block copolymer, can also be prepared by atom transfer radical polymerization (“ATRP”). Controlled polymerization of N-isopropylacrylamide (NIPAAM) by atom (ATRP) can be effected using ethyl 2-chloropropionate (ECP) as initiator and CuCl/tris(2-dimethylaminoethyl)amine (Me₆TREN) as a catalytic system. The living character of the polymerization allows preparation of block copolymers.

Aqueous solutions of graft copolymer I will phase-separate and form a gel above a lower critical solution temperature (LCST). Typically, this phase separation results in rapid deswelling, loss of entrapped water, and rapid uncontrolled drug release upon gelation. Graft copolymer I, as described herein above, comprises a thermosensitivity imparted by the main polyacrylamide polymer chain and swelling controlled independently by the graft polyether chains with a small effect on polymer LCST.

Gels were formed by dissolving various embodiments of Applicants' graft copolymer I in 150 mM phosphate buffered saline (pH 7.4) at between 5 and 45 wt % polymer in water. For controlled release of drugs or proteins, the desired amount of drug or protein can be directly added to the polymer solution below the graft copolymer LCST (such as at room temperature) either as a solution or suspension.

Examples of applications of these materials include protein release (such as release of rhBMP2 for accelerated bone healing) or for in situ space-filling use such as embolization or as a contraceptive. The hydrolytic stability, hydrophilicity, and minimal LCST effect inherent in JAAm, monomer IX wherein the water soluble polymer comprises polyether IV wherein p is about 3 and wherein r is about 19 and wherein R6 is methoxy, make this monomer suitable for inclusion in a variety of temperature responsive biomaterials where swelling control or controlled delivery of hydrophilic drugs are desired.

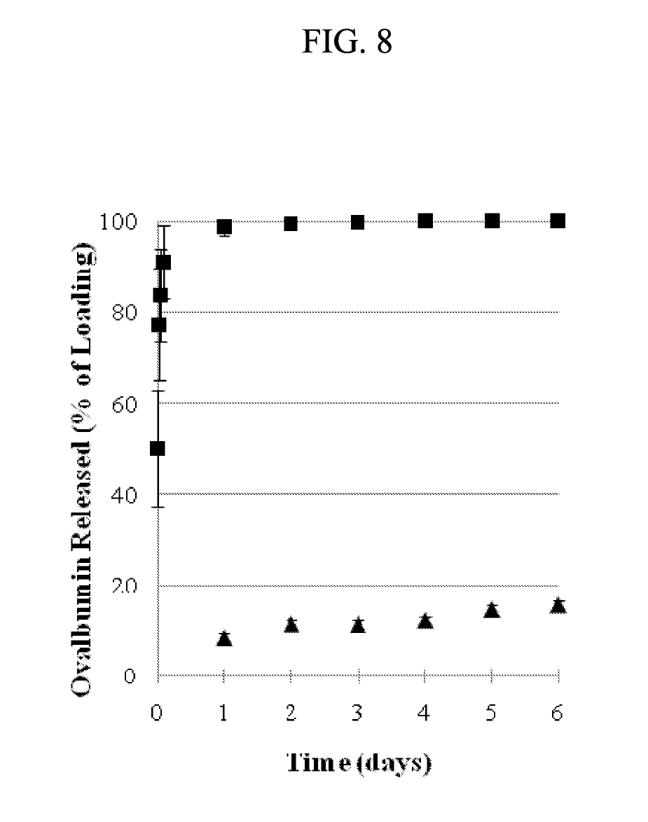

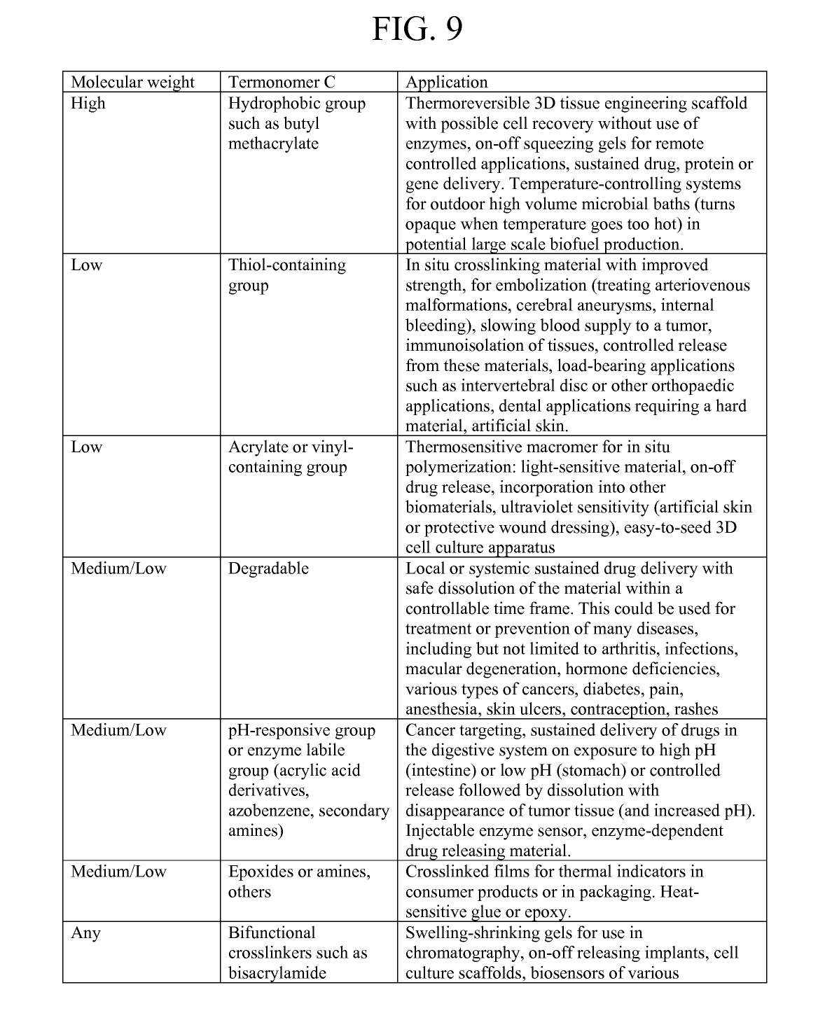

In certain embodiments, Applicants' polymeric hydrogel comprises a terpolymer, wherein monomers VII and IX are polymerized with a termonomer C. FIG. 8 summarizes certain polymeric terpolymer hydrogels formed by polymerizing NIPAAM and JAAm in combination with a termonomer C.

The following examples are presented to further illustrate to persons skilled in the art how to make and use the invention. These examples are not intended as a limitation, however, upon the scope of the invention.

### EXAMPLES

Temperature-responsive graft copolymers of N-isopropylacrylamide (NIPAAm) with JEFFAMINE M-1000 acrylamide (JAAm) were synthesized by radical polymerization to form graft copolymer I wherein in R3 and R4 are methyl.

All materials were reagent grade and obtained from Sigma-Aldrich unless otherwise noted. NIPAAm monomer was recrystallized from hexane. Azobisisobutyronitrile (AIBN) was recrystallized from methanol. Benzene and 1,4-dioxane were anhydrous and used as received. HPLC grade tetrahydrofuran (THF) was used for low molecular weight polymerizations and as the mobile phase for molecular weight and polydispersity determination. JEFFAMINE M-1000 polyetheramine was donated by Huntsman Corporation (The Woodlands, Tex., USA).

JEFFAMINE M-1000 acrylamide (JAAm) monomer was synthesized from JEFFAMINE M-1000 polyetheramine. JEFFAMINE M-1000 (20 g, 20 mmol) was dissolved at 10 w/v % in dichloromethane (DCM) along with triethylamine (3.3 mL, 24 mmol) and maintained at 0° C. under nitrogen atmosphere. Acryloyl chloride (1.95 mL, 24 mmol) was then added dropwise into the solution under stirring and the reaction was allowed to proceed for at least 6 hours at 0-4° C. at under nitrogen atmosphere. Following the reaction, DCM was evaporated and the residue was dissolved in 0.1 N NaHCO3 (200 mL). The product was extracted into DCM and the organic layer evaporated once more. JAAm was solidified by cooling on ice, vacuum dried, and stored at 4° C.

Poly(NIPAAm-co-JAAm) copolymers were synthesized by radical polymerization in each of two solvent mixtures, either 90:10 benzene:dioxane (high molecular weight, HMW) or 80:20 dioxane:THF (low molecular weight, LMW), as shown in Scheme 1B. Feed ratios in the polymerizations were either 100:0, 85:15, or 70:30 NIPAAm:JAAm by mass. Monomer solutions were bubbled with nitrogen for at least 20 minutes prior to addition of the initiator to reduce dissolved oxygen. Polymerizations were conducted at 65° C. for 24 hr under a slight positive pressure of nitrogen, with AIBN (0.007 mol AIBN/mol of total monomer) as the initiator. For HMW polymerizations only, approximately half of the solvent was either decanted or evaporated and then replaced by an equivalent volume of acetone to reduce the viscosity of the polymer solution. Copolymers were collected by precipitation in 10-fold (HMW) or 15-fold (LMW) excess of chilled diethyl ether, filtered, and vacuum-dried overnight. The product was then dissolved in deionized water, dialyzed against either 10,000 MWCO (HMW) or 3,500 MWCO (LMW) at 4° C. for at least 3 days, and lyophilized to obtain the poly(NIPAAm-co-JAAm) polymers.

The polymer feed ratios, composition, molecular weight, and LCST as measured by DSC are shown in Table 1.

TABLE 1
Composition, molecular weight distribution, and
LCST of poly(NIPAAm-co-JAAm) copolymers

| | JAAm content (wt %) | | M_{w} | Pd | |
|---|---|---|---|---|---|
| Polymer | Feed Ratio | Composition | (×10³ Da) | (M_{w}/Mₙ) | LCST (° C.) |
| H 0 | 0 | 0 | 861.0 | 1.90 | 27.83 ± 0.06 |
| H 15 | 15 | 11.9 | 226.6 | 1.84 | 31.07 ± 0.06 |
| H 30 | 30 | 22.4 | 229.1 | 1.67 | 33.87 ± 0.15 |
| L 0 | 0 | 0 | 30.0 | 2.03 | 29.6 ± 0.06 |
| L 15 | 15 | 12.1 | 28.8 | 2.02 | 32.4 ± 0.06 |
| L 30 | 30 | 24.2 | 37.2 | 2.02 | 35.4 ± 0.06 |

Polymers are classified in terms of their molecular weight (H for high, L for low) and JAAm fraction in the feed (0, 15, or 30 wt %). When applicable, polymer concentration is written before the molecular weight (i.e. 20 H 30). LMW polymers all had a polydispersity near 2.0 and Mw between 28.8 and 37.2 kDa. HMW poly(NIPAAm) had a weight-average molecular weight (Mw) of 861 kDa, while the molecular weights of both HMW copolymers containing JAAm were considerably lower with Mw near 230 kDa. Polydispersities of HMW copolymers were slightly lower than those of LMW polymers, ranging from 1.67 to 1.90.

1H Nuclear Magnetic Resonance (1H NMR). 1H NMR (Varian Inova, 300 MHz) was used to confirm successful synthesis and determine the chemical composition of the synthesized polymers. D2O was used as the NMR solvent.

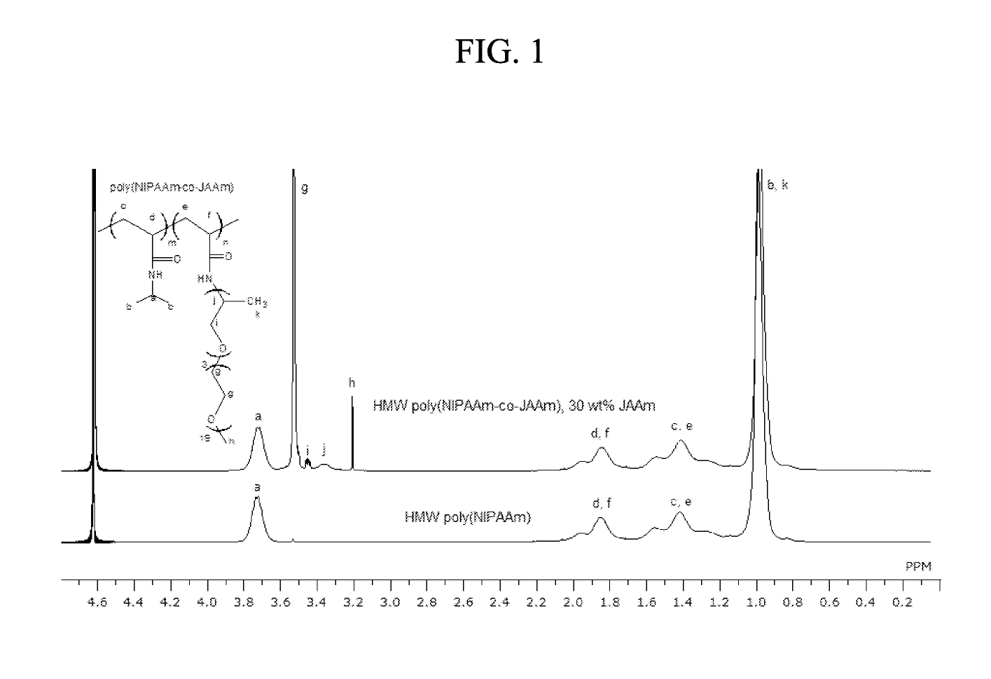

Successful synthesis of the copolymers was confirmed by ¹H NMR as shown in FIG. 1. Referring now to FIG. 1, JAAm content in the copolymers was calculated from the integration ratios of the peak at 3.5 ppm ascribed to the oxyethylene protons of the EO units (CH₂CH₂O) of JAAm relative to the peak at 3.7 ppm (1H) of the lone isopropyl proton of NIPAAm (CH(CH₃)₂). JAAm has an average of about 75 EO protons given an average molecular weight of 1,054 g/mol (calculated based on 1,000 g/mol for JEFFAMINE M-1000). For both low and high molecular weight copolymers, a feed ratio by weight of 85:15 NIPAAm:JAAm produced a copolymer with similar composition to feed. Synthesis with feed ratios of 70:30 led to slightly lower incorporation of JAAm—22.4 wt % for H 30 and 24.2 wt % for L 30. JAAm content in the copolymers is reported as wt % in this work because the weight fraction of PEG grafted onto a poly(NIPAAm) backbone determines equilibrium swelling rather than the molar fraction.

The molecular weight and polydispersity of the synthesized polymers was determined by gel permeation chromatography (Shimadzu Corporation) in conjunction with static light scattering (MiniDawn, Wyatt Technology Corporation). Samples were prepared by dissolving the polymers in THF with a concentration of 10 mg/mL.

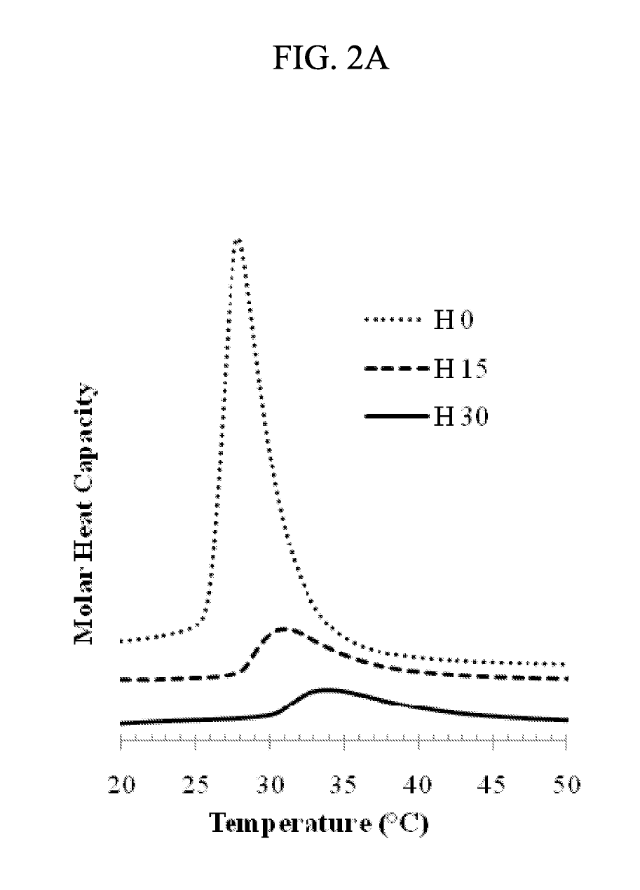

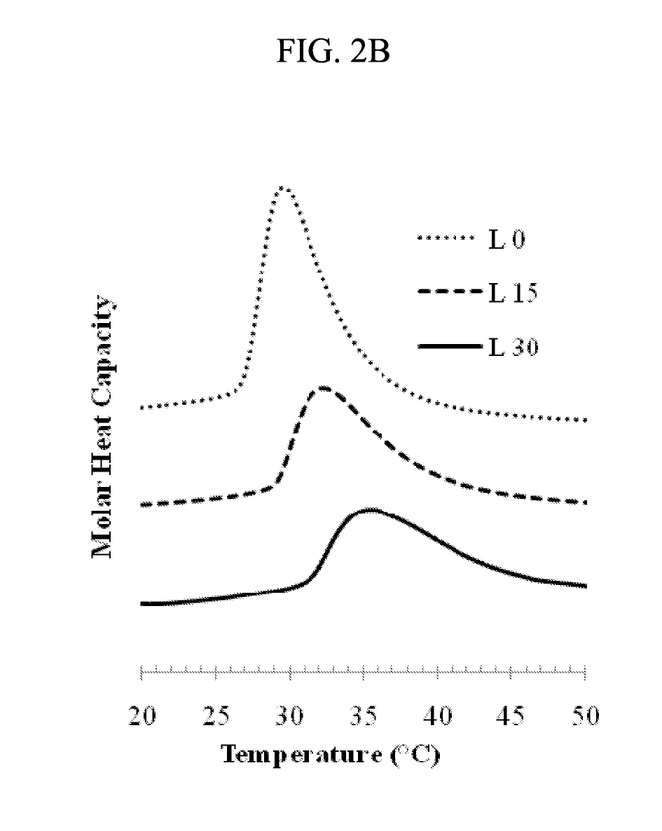

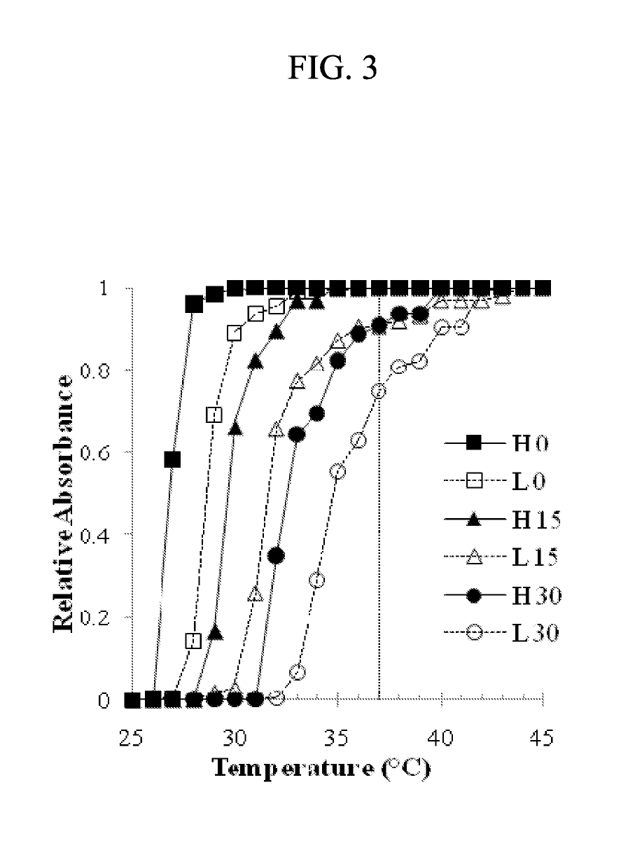

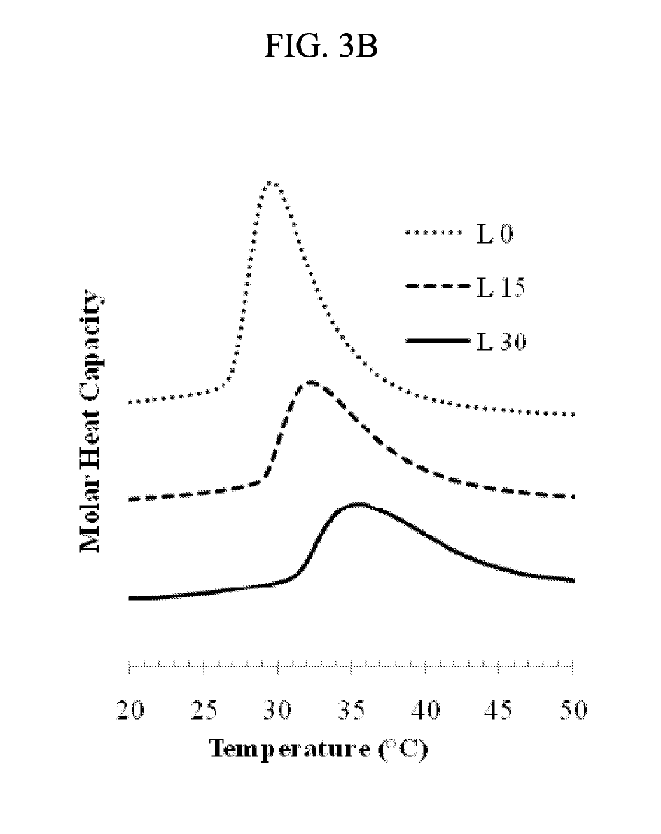

The LCST transition of each polymer at 5 wt % in PBS was characterized by DSC as shown in FIG. 2. Samples were dissolved at 5 wt % in 150 mM PBS (pH 7.4). Scans were taken from 10° C. to 80° C. at a heating rate of 1° C./min. Samples were measured in triplicate.

For both HMW and LMW copolymers, increasing JAAm content in the polymer caused an increase in the material LCST, which is consistent with previously reported data for copolymers of NIPAAm with PEG acrylates or methacrylates. The onset of the transition for each molecular weight range is about 5° C. higher for H 30 and L 30 polymers compared to the respective homopolymers. Increasing JAAm content also leads to broadening of the LCST endotherm. HMW homopolymer has a greater enthalpy of gelation (area under the curve) than either H 15 or H 30. This is likely due to two factors. First, the energy of the phase transition decreases as the temperature of that transition increases, as has been shown before for other NIPAAm-based polymers. Second, the average molecular weight of H 0 is much larger than H 15 or H 30, and more energy is required to cause the coil-to-globule transition of a higher molecular weight polymer chain.

Cloud Point Determination. Synthesized copolymers were dissolved at 0.1 wt % in 150 mM PBS (pH 7.4) and analyzed for LCST properties by cloud point determination. This concentration was chosen because none of the polymer solutions saturated the detector when heated above the LCST. Cuvettes containing the polymer solutions were allowed to equilibrate in a water bath for at least 90 s prior to each measurement. Absorbance at 450 nm was measured every 1° C. by a UV/Vis spectrometer from 25-45° C. with buffer alone as the reference. Some polymers precipitated and formed aggregates upon heating. In this case, the last value of absorbance before observed aggregation was recorded as the maximum value and all previous values were normalized relative to the maximum value. Absorbance values for polymers that did not aggregate were normalized to the absorbance at 55° C.

The swelling behavior and gel stability of the synthesized copolymers was characterized at various concentrations and molecular weights. Solutions of each low molecular weight (LMW) polymer were prepared at 5, 10, 20, and 30 wt % and of each high molecular weight (HMW) polymer at 5, 10, and 20 wt %. Solutions of HMW polymers at 30 wt % were very viscous and difficult to dispense, particularly for homopolymer.

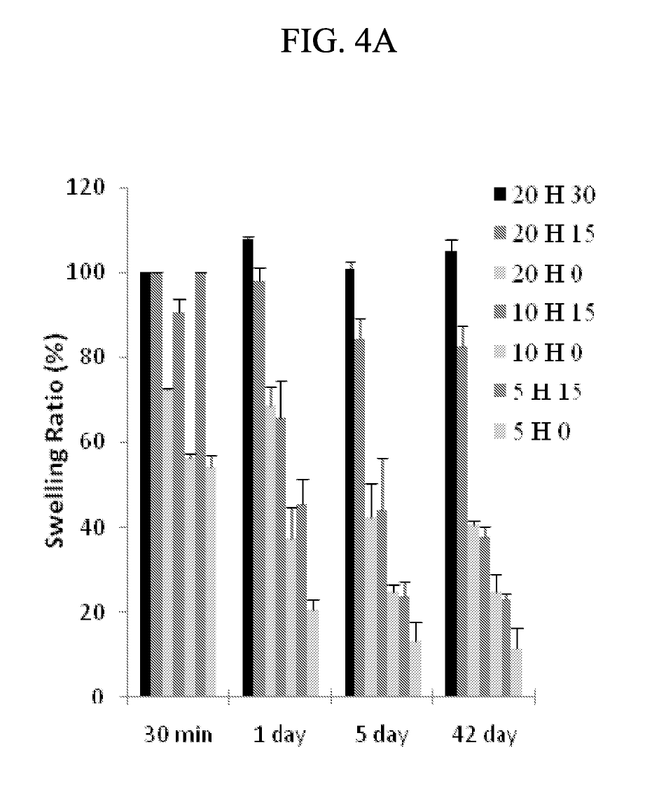

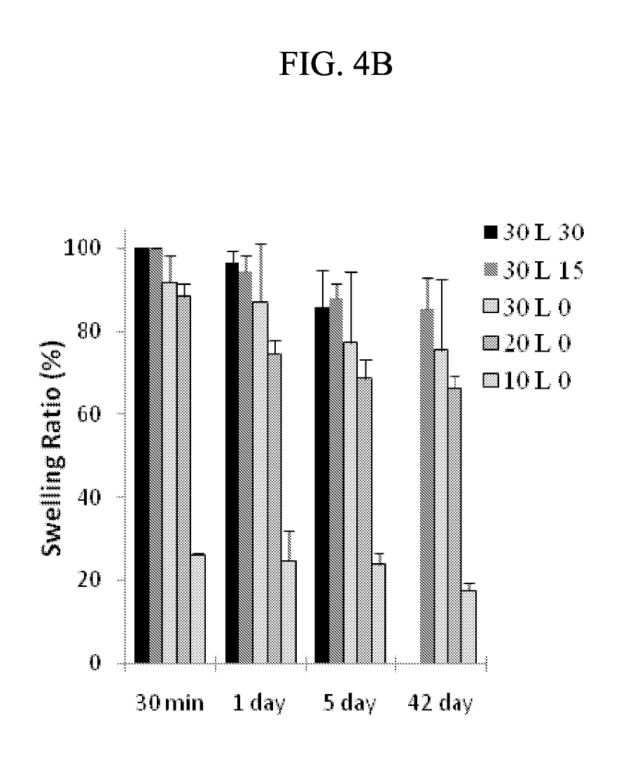

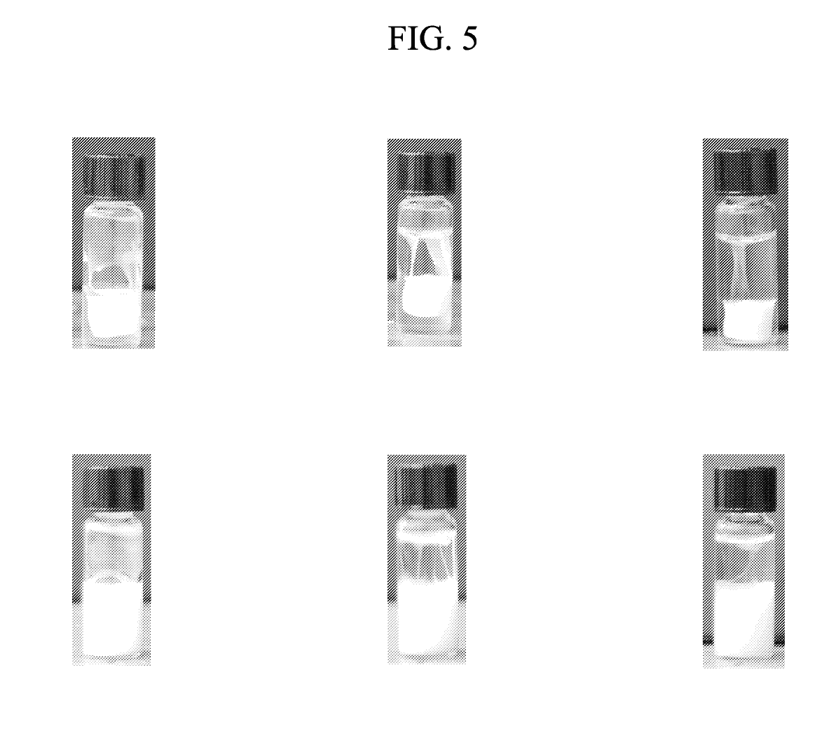

FIG. 4 shows the gelation and swelling behavior of those HMW polymer solutions which formed opaque gels after 5 days. FIG. 5 shows the gelation and swelling behavior of LMW polymer solutions. The difference in gel formation between H 15 and H 30 demonstrates that the critical polymer concentration required to form a gel increases with JAAm content at a given molecular weight. The hydrophilic, EO-rich, JEFFAMINE M-1000 grafts in these materials hinder the association of hydrophobic core regions within the solution when heated above its LCST, and therefore greater polymer concentration is necessary to form a physical gel.

In cases where HMW polymer solutions formed stable gels, those with greater JAAm incorporation underwent less and slower syneresis on average. Homopolymer gels with low equilibrium swelling ratios began to expel a substantial fraction of PBS within 30 minutes of heating above the LCST while gels with JAAm and similarly low equilibrium swelling retained the initially entrapped water for hours after gelation. This difference in initial shrinking rate indicates that JAAm incorporation leads to greater friction between the ejected liquid and the gel. Afterwards, the gels tended toward equilibrium over another 2-5 days due to a slower rearrangement process during which local contacts between polymer molecules interchange to become increasingly favorable. Statistically significant differences (α=0.05) in swelling ratio due to JAAm inclusion after 42 days were observed at 5 wt % between 5 H 0 and 5 H 15 (p=0.014), at 10 wt % between 10 H 0 and 10 H 15 (p=0.009), and at 20 wt % between each pair of sample groups (20 H 0 vs. 20 H 15, 20 H 15 vs. 20 H 30, and 20 H 0 vs. 20 H 30) (all p<0.005). While 20 H 30 was the only solution with 30% JAAm in the feed that yielded gels at 37° C., those gels exhibited excellent resistance to shrinking and stability under physiological conditions, maintaining approximately 105% of their original volume after 42 days. Accordingly, 20 H 15 gels underwent minimal syneresis over 5 days (83% of original volume), and 20 H 0 homopolymer gels collapsed to a much greater extent, decreasing to about 42% of their original volume over 5 days. Representative gels of 20 H 0 and 20 H 30 are shown at various times after gelation in FIG. 5.

Three approximately 1 g aliquots of each polymer solution were placed into each of three 2 mL glass vials and heated to 37° C. in a water bath. After 30 minutes, vials were photographed and then 1 mL of 37° C. pre-warmed PBS was added to each sample. Solutions were maintained in a 37° C. room for the remainder of the study. Vials were photographed at various time points to assess gel swelling. Images of the vials were cropped to contain only the entire water volume in the vial. Images for each vial at each time point were converted to grayscale and then thresholded into either white (gel) or black (not gel) pixels both manually and using MATLAB. Manual thresholding was done to remove image artifacts such as light reflections and thin polymer films from vials. The initial gel height in pixels corrected for any differences in image size was calculated for each sample in MATLAB using the equation hgel, i=wt (hi/wi), where wt is the width in pixels of the image at time t, and hi and wi are the height and width, respectively, in pixels of the image of a sample (gel plus any expelled water) after gelation but before any additional buffer was placed on top of the gels. Gel volume was determined by assuming that horizontal cross sections of each gel were circular. The number of white (gel) pixels in each row of an image were calculated, then each row's pixel count divided by 2 and squared. The sum of these values is a measure of volume, Vgel,t. The initial gel volume for the same sample, Vgel,i, was determined using the formula Vgel,i=hgel,i (wt/2)². Swelling was then reported as a fraction of the initial gel volume, i.e. Swelling=Vgel,t/Vgel,i.

Copolymers with low molecular weight in general had much poorer gelation characteristics, as shown in FIG. 4B. Polymer solutions not shown in the legend of FIG. 4B separated into a small translucent or opaque phase and settled on the bottom of the vials within two hours upon heating to 37° C. Homopolymer solutions at 10 wt % and above formed gels at 37° C., while polymers containing JAAm only formed gels at 30 wt %. The lack of gelation observed in copolymers containing JAAm is likely due to a combination of two factors. First, the transition temperatures of L 15 and L 30 are both higher and more broadly distributed than HMW polymers with similar composition, and so fewer chains are insoluble at 37° C. Second, low molecular weight polymers require greater concentrations to form gels above the LCST as opposed to milky solutions or precipitates, as even poly(NIPAAm) did not form gels at 5 wt % at a similar molecular weight to the other LMW polymers.

Equilibrium swelling of homopolymer gels increased with polymer concentration, with 30 L 0 having an equilibrium swelling ratio of 75%. While 30 L 30 and 30 L 15 gels retained a greater swelling ratio on average than 30 L 0 for the first 3 days, the swelling ratios between any pair of these gels was not significantly different at any time point. At 42 days, 30 L 30 became translucent and flowed when inverted, so it was not considered a gel at that time, perhaps due to slow dissolution of the polymer or sensitivity to small temperature fluctuations (as low as 35° C.) during incubation.

Copolymers of NIPAAm with hydrophilic comonomers have a tradeoff between the fraction of comonomer to control shrinking and the polymer concentration required to form a stable gel. Within this low molecular weight range (Mw 28-38 kDa), incorporation of JAAm causes the disadvantages of this tradeoff to overlap such that the concentration required to form a gel is so high that the homopolymer gels have equilibrium swelling similar to that of copolymer gels. However, JAAm may provide controlled shrinking and drug delivery properties to more hydrophobic polymers in this molecular weight range. In particular, we have previously developed resorbable materials with initial LCST below 25° C. in the 30-40 kDa range which undergo substantial shrinking even at high concentrations.

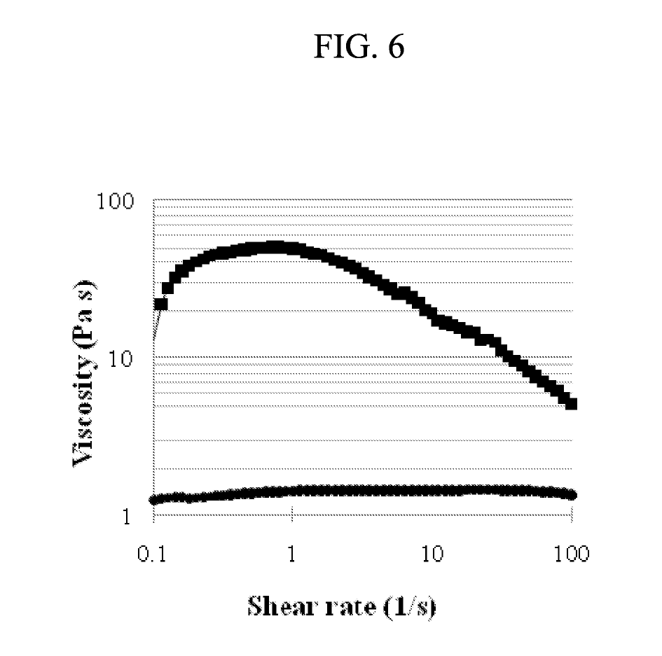

Selected polymer solutions 20 H 0 and 20 H 30 were characterized for their viscosity in the sol phase and mechanical properties in the gel phase by parallel plate rheometry. Solution viscosity versus shear rate is shown in FIG. 6. Homopolymer solution is shear-thinning above 1 Hz while copolymer solution is approximately Newtonian over the range of shear rates tested. At 1/s shear rate, 20 H 0 was about 35 times more viscous than 20 H 30. This difference can be attributed to both the higher molecular weight of the homopolymer and the JAAm content. The 20 H 30 polymer solution tended to flow and was easy to handle in the sol phase.

The mechanical properties of 20 wt % HMW poly(NIPAAm) (20 H 0) and 20 wt % HMW poly(NIPAAm (70 wt %)-co-JAAm (30 wt %)) (20 H 30) were measured by rheometry in both the sol and gel states. For each run, about 400 uL of polymer solution was placed between the flat 25 mm plates of a rheometer (Anton Paar MCR-101), with a gap height of approximately 0.5 mm. Viscosity of the copolymer solutions was evaluated at 20° C. under continuous rotation of the top plate for shear rates 0.1-100 l/s. Gels were evaluated at 37° C. with the normal force maintained during measurement at 100+/−50 mN and a humidity chamber placed over the sample to reduce evaporation.

Polymer solutions were placed on the rheometer at 20° C. and then heated to 37° C. for 60 seconds before measurements were taken. The linear viscoelastic region for each polymer solution was determined by varying the oscillatory strain applied to the gels between 0.01% and 25% at 1 Hz frequency (not shown). The materials were then subjected to oscillatory strain within the linear viscoelastic region (0.5% strain for gels with JAAm, 1% strain for gels without JAAm) and the frequency varied from 0.1 to 100 Hz (n=4). Mean and deviation of storage modulus (G′) and loss modulus (G″) were determined for each data point. Deviation was calculated using log-transformed data.

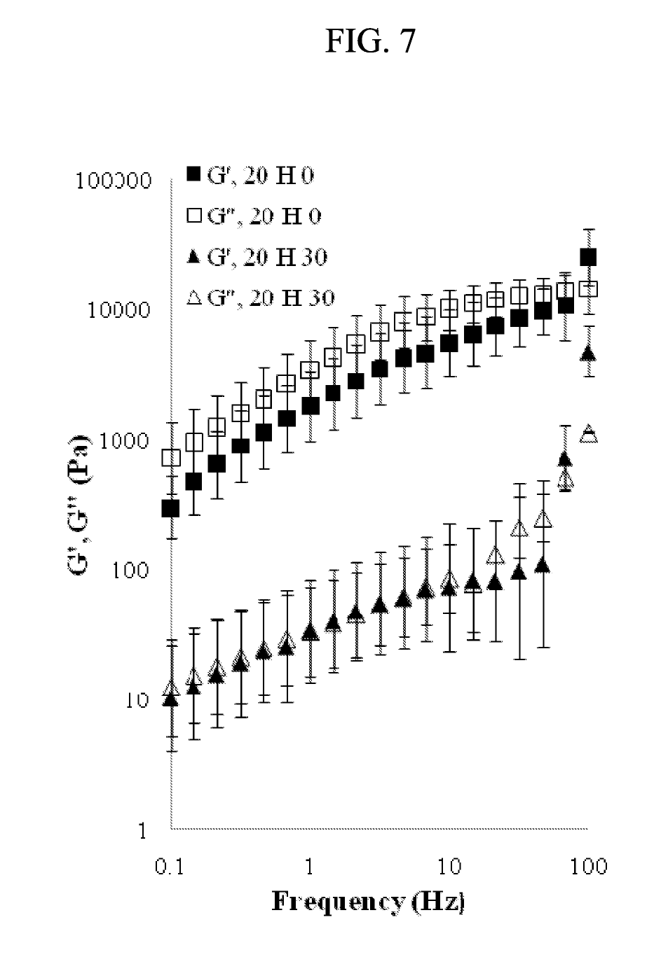

Frequency-dependent storage and loss moduli of 20 H 0 and 20 H 30 under oscillatory strain at 37° C. are shown in FIG. 7. Each gel exhibits increased resistance to deformation at higher frequencies which is characteristic of physical gels.12 In the frequency range 0.1-10 Hz, both gels have a phase angle near 45° (i.e. G′=G″) which is characteristic of a viscoelastic material.

Homopolymer gels have both storage and loss moduli in the 0.1-10 kPa range, while the moduli for copolymer gels are lower, in the 10-100 Pa range on average. Gels with JAAm are weaker than homopolymer gels mostly due to their lower molecular weight, higher water content, and incomplete LCST transition at 37° C. The latter could be addressed by fractionation in aqueous medium or by incorporating a hydrophobic comonomer such as butyl methacrylate into the polymer. Although the samples used in this study were analyzed one minute after heating to body temperature, 20 H 0 gels expelled water within that time as well as during the course of the measurements while 20 H 30 gels did not.

Syneresis of the 20 H 0 gels was exacerbated by the high surface area to volume ratio of these gels on the rheometer stage, so that by the end of the measurement time (about 5 minutes), the gels had lost much of their volume. Gels of 20 H 0 that were heated for a longer time prior to measurement exhibited moduli higher than those shown in FIG. 7 by up to 2 orders of magnitude (data not shown). In terms of their potential for use as biomaterials, copolymers with JAAm maintain consistent properties after gelation and lower resistance to deformation whereas homopolymer gels have highly dynamic properties but higher resistance to deformation.

Protein release kinetics from 20 wt % HMW poly(NIPAAm) and 20 wt % HMW poly(NIPAAm (70 wt %)-co-JAAm (30 wt %)) hydrogels were measured at 37° C. using ovalbumin (MW ˜44.3 kDa) as a model drug. Ovalbumin (10 mg/mL) was dissolved in the polymer solutions at 4° C. and then 1 g samples (n=3) were weighed out of each common solution into 4 mL vials. Gels were formed by incubation in a 37° C. water bath for 15 minutes. The 4 mL vials with gels were then inserted into pre-warmed 20 mL vials which were then filled to the top with 20 mL pre-warmed PBS and maintained in a 37° C. room. Buffer was completely replaced for homopolymer samples after 1 day incubation in order to maintain infinite sink conditions. Aliquots were taken at various time points and frozen at −20° C. Protein concentration in the aliquots was measured at the end of the study using the BCA Protein Assay (Pierce Biotechnology, Rockford, Ill.) according to the manufacturer's instructions using a UV/Vis spectrophotometer (BMG Labtech Fluostar Omega).

Release kinetics of ovalbumin from 20 H 0 and 20 H 30 gels at 37° C. is shown in FIG. 8. Homopolymer gels provided fast release. During the first 15 minutes after gelation, gels decreased in volume by only about 20%, yet over 50% of the loaded ovalbumin was released in the same time. Over ninety percent of the loaded ovalbumin was released within 3 hours. On the other hand, release from the 20 H 30 gels was much slower. Only 8% release was observed within one day after gelation, and an additional 7% was released over the following 5 days. The lack of high initial burst release from 20 H 30 gels can be attributed to resistance to syneresis. However, the slow rate of release over a period of several days indicates that the diffusivity of ovalbumin is greatly reduced in gels containing JAAm.

The lack of high initial burst release from 20 H 30 gels can be attributed to resistance to syneresis. Upon heating above the LCST, the polymer solution phase-separates into two phases—a homogeneous polymer-rich gel phase—consisting of nearly all of the polymer plus some fraction of water—and a nearly pure solvent phase. For 20 H 30 gels, the equilibrium water content of the gel being near the initial content led to a phase transition with minimal phase separation—therefore it can be assumed the protein is retained almost entirely within the polymer-rich gel phase. As the rate of release from non-crosslinked physical gels is known to be inversely related to their viscosity, it follows that the high viscosity of the 20 H 30 gel phase combined with little to no phase separation is the primary cause of the slow and sustained release of ovalbumin observed. Conversely, the phase transition of homopolymer gels leads to a high degree of phase separation following gelation, resulting in a hydrophobic polymer-rich gel phase and excess water. A possible explanation for the rapid albumin release is that, after phase separation, the albumin preferentially dissolved (partitioned) into the excess water phase based on its hydrophilicity and therefore rapidly diffused from the homopolymer gels. The vast difference in protein release kinetics from the two polymers used in this study demonstrates the potential utility of these materials for controlled drug delivery applications.

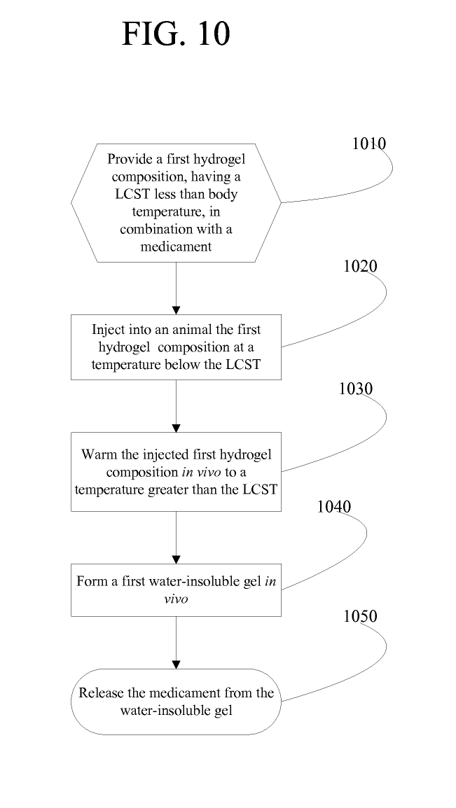

FIG. 10 summarizes Applicants' method to deliver a medicament to an injection site within the body of an animal, including a human. In certain embodiments, the injection site comprises the surface of an orthopaedic implant. In certain embodiments, the injection site comprises the surface of a bone. In certain embodiments, the injection site comprises a joint space. In certain embodiments, the injection site comprises the peritoneum. In certain embodiments, the injection site comprises a subcutaneous injection.

By medicament, Applicants mean a material selected from the group consisting of a Nucleic acid, a Protein (including growth factors, bone morphogenetic proteins), a Polypeptide, a Contrast agent for imaging, an Anesthetic, an Antineoplastic agent, an Antifungal, an Anti-inflammatory drug (steroids, non-steroidal anti-inflammatory drugs (NSAIDs), and an Antibiotic. In certain embodiments, the Antibiotic comprises one or more of Aminoglycosides, including gentamicin, amikacin, and tobramycin, Cephalosporins including cefazolin, Vancomycin, and Rifampin.

Referring now to FIG. 10, in block 1010 the method provides a medicament and a hydrogel comprising a LCST less than the body temperature of a subject animal. Those skilled in the art will appreciate, that body temperature for a human is about 37° C. In these human injection embodiments, the hydrogel of step 1010 comprises a LCST less than about 37° C.

In certain embodiments, the hydrogel of step 1010 comprises a polymeric material comprising a backbone formed from one or more substituted acrylamides in combination with pendent polyether chains grafted onto the polymeric backbone.

In certain embodiments, the hydrogel of block 1010 comprises Applicants' hydrogel I. In certain embodiments, the hydrogel of block 1010 comprises Applicant's hydrogel formed from a copolymer comprising N-isopropylacrylamide and JEFFAMINE M-1000 acrylamide.

In block 1020, an aqueous solution the medicament and the hydrogel of block 1010 is injected into a selected animal at a temperature less than the LCST. In certain embodiments, the injection site comprises a tissue space wherein a subsequently formed gel will substantially completely fill that tissue space. In certain embodiments, the hydrogel of block 1010 is utilized in conjunction with implantation of an artificial joint. In certain of these embodiments, the injection of block 1020 is performed after implantation such that the injected hydrogel is disposed adjacent a surface of the implanted artificial joint.

In certain embodiments, the hydrogel of block 1010 is coated onto a surface of an artificial joint prior to implantation. In these embodiments, the “injection” of block 1020 comprises implantation of the artificial joint comprising a surface coated with the hydrogel of block 1010.

In block 1030, the hydrogel of block 1010 injected into the body of an animal in block 1020 is warmed in vivo to a temperature greater than the LCST. In certain embodiments, the warming of block 1030 is performed by the body heat of the animal. In certain embodiments, the warming of block 1030 is performed by disposing a heated object, such as for example and without limitation, a heating pad, hot compress, and the like, onto the skin of the animal in near proximity to the injection site. In certain embodiments, the waring of block 1030 is performed using a heat lamp.

In block 1040, the hydrogel of block 1010 injected into the body of an animal in block 1020 and warmed in vivo to a temperature greater than the LCST in block 1030, forms in vivo a water-insoluble gel. In certain embodiments, the water-insoluble gel of block 1040 is formed in, and substantially fills, a tissue space. In certain embodiments, the water-insoluble gel of block 1040 is disposed on, and in near vicinity to, a surface of a joint implant.

In block 1050, the water-insoluble gel of block 1040 releases the medicament of block 1010 into tissues adjacent the injection site of block 1020. In certain embodiments, the medicament is released at a substantially uniform rate over time. In certain embodiments, the aggregate amount of medicament released from the water-insoluble gel of block 1040 into the injection site of block 1020 plotted on a Y axis of a graph against time plotted on an X axis of the graph over time can be approximately modeled by a linear equation of the type y=mx+b, wherein m is slope of the straight line and b is the intercept of the straight line with the Y axis. As a general matter, the intercept is 0.

In other embodiments, the release is approximately proportional to the square root of time over the first 60% of release, with a slower rate of release thereafter.

While the preferred embodiments of the present invention have been illustrated in detail, it should be apparent that modifications and adaptations to those embodiments may occur to one skilled in the art without departing from the scope of the present invention as set forth in the following claims.

1. A hydrogel, comprising:
a polymer backbone comprising a plurality of repeat units, wherein one or more of the plurality of repeat units comprises a pendent water soluble polymer attached thereto by a linkage selected from the group consisting of amide, thioamide, urea, and thiourea;
wherein:
an aqueous solution of said polymer comprises a lower critical solution temperature (“LCST”);
said LCST is less than 37° C.

2. The hydrogel of claim 1, further comprising a first plurality of repeat units having a structure:

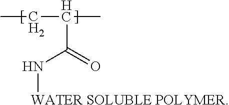

3. The hydrogel of claim 1, further comprising a first plurality of repeat units having a structure:

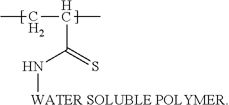

4. The hydrogel of claim 1, further comprising a first plurality of repeat units having a structure:

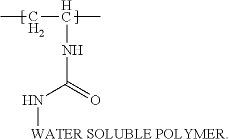

5. The hydrogel of claim 1, further comprising a first plurality of repeat units having a structure:

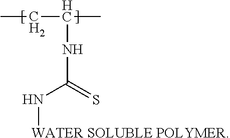

6. The hydrogel of claim 1, wherein said polymer backbone comprises a copolymer.

7. The hydrogel of claim 6, wherein said copolymer comprises a second plurality of repeat units having a structure: 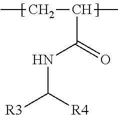
wherein R3 and R4 are each independently selected from the group consisting of H, methyl, ethyl, and phenyl.

8. The hydrogel of claim 7, wherein the water soluble polymer comprises a polyether.

9. The hydrogel of claim 8, wherein the polyether comprises a plurality of third repeat units having a structure:

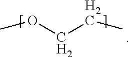

10. The hydrogel of claim 8, wherein the polyether comprises a third plurality of repeat units having a structure:

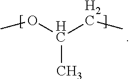

11. A method to deliver a medicament in vivo, comprising:
forming an aqueous solution comprising a hydrogel and a medicament, wherein said hydrogel comprises a lower critical solution temperature (“LCST”), and further comprises a polymer backbone comprising a plurality of repeat units, wherein one or more of the plurality of repeat units comprises a pendent water soluble polymer attached thereto; wherein:
the temperature of said aqueous solution is less than said LCST;
injecting at an injection site said aqueous solution into the body of an animal, including a human, said animal comprising a body temperature, wherein said LCST is less than said body temperature;
warming in vivo said aqueous solution to said body temperature;
forming in vivo a water-insoluble gel at said injection site; and
releasing in vivo said medicament from said water-insoluble gel.

12. The method of claim 11, wherein said hydrogel further comprises a first plurality of repeat units having a structure:

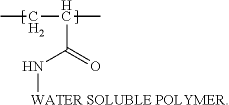

13. The method of claim 11, wherein said hydrogel further comprises a first plurality of repeat units having a structure:

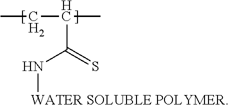

14. The method of claim 11, wherein said hydrogel further comprises a first plurality of repeat units having a structure:

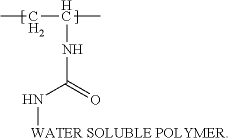

15. The method of claim 11, wherein said hydrogel further comprises a first-plurality of repeat units having a structure:

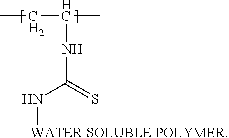

16. The method of claim 11, wherein said hydrogel comprises a copolymer.

17. The method of claim 16, wherein said copolymer comprises a second plurality of repeat units having a structure: 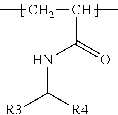
wherein R3 and R4 are each independently selected from the group consisting of H, methyl, ethyl, and phenyl.

18. The method of claim 17, wherein the water-soluble polymer comprises a polyether.

19. The method of claim 18, wherein the polyether comprises a plurality of third repeat units having a structure:

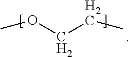

20. The method of claim 18, wherein the polyether comprises a third plurality of repeat units having a structure: [chemical structure]
